(19) United States
(12) Patent Application Publication
(10) Pub. No.: US 2013/0053804 A1
(43) Pub. Date: Feb. 28, 2013

(54) DEVICE FOR ATTACHMENT TO A PENIS

(71) Applicants: Nicolai Sørensen, Hellebaek (DK); Brian Nielsen, Gorlose (DK)

(72) Inventors: Nicolai Sørensen, Hellebaek (DK); Brian Nielsen, Gorlose (DK)

(73) Assignee: Securin ApS, Hillerød (DK)

(21) Appl. No.: 13/576,700

(22) PCT Filed: Feb. 11, 2011
(86) PCT No.: PCT/EP2011/052066

§ 371 Date: Oct. 31, 2012

(30) Foreign Application Priority Data
Feb. 13, 2010 (DK) PA 2010 00123
Apr. 5, 2010 (DK) PA 2010 00279

(51) Int. Cl. A61F 5/453

(52) U.S. Cl. 604/349

(57) ABSTRACT

An attachment member suitable for attaching a system to a male human being's penis, said system being suitable for collecting urine from a male human being, said attachment member comprising a substantially cylindrical portion which: comprises an elastic film material, has a first opening at a first end thereof and a second opening at the other end thereof, is arranged such that at least the glans penis of a penis could be arranged inside the substantially cylindrical portion with the first opening being further from the base of the penis than the second opening, and is at least partly rolled up to assume a ring like configuration in such a way that the first opening is located inside the rolled up portion and the second opening is located outside the rolled up portion, wherein the inner diameter of the rolled up portion of the substantially cylindrical portion is less than 45 mm when the elastic film material of the attachment member is not stretched and not rolled up. In this way, an effective and simple to apply attachment member is provided.

### FIELD OF THE INVENTION

The present invention relates to an attachment member for attaching a urine collecting system to a penis and a urine collecting system.

More specifically, the present invention relates to an attachment member suitable for attaching a system for collecting urine from a male human being to a male human being's penis, the attachment member comprising a substantially cylindrical portion comprising an elastic film material, having a first opening at a first end thereof and a second opening at the other end thereof, being arranged such that at least the glans penis of a penis could be arranged inside the substantially cylindrical portion with the first opening being further from the base of the penis than the second opening, and being at least partly rolled up to assume a ring like configuration in such a way that the first opening is located inside the rolled up portion and the second opening is located outside the rolled up portion.

Furthermore, the present invention relates to a method of applying an attachment member to a penis and a method of applying a urine collecting system and an attachment member to a penis.

### BACKGROUND OF THE INVENTION

Urine collecting systems for males can be divided into two main types, internal systems which make use of a catheter or other implement inserted into the urea and external systems where a urisheath is attached to the outside of the penis.

A known problem with external urine collecting systems is that they frequently drop off the penis as the urisheath fails to stay in place. Furthermore a person skilled in the art knows that it can be difficult to apply urisheaths on many patients, for example elderly and overweight persons, as the penis of many patients can be fairly small, can have a non-firm form and can contain wrinkled skin.

All known urisheaths are provided in a rolled up configuration and are applied near the exit of the urea and rolled onto the penis in a direction towards the base of the penis in a manner similar to a condom. Most of the time this procedure is difficult due to the above described nature of the penis.

Many have tried to solve these issues by improving the flexibility of the urisheath, applying adhesives on the urisheath, optimizing the fit of the urisheath to the penis and building teaching materials for nurses on how to apply urisheaths.

EP0068712 and FR2752158 try to solve these problems by using a flexible condom-like material and by using an applicator. However they do not overcome the problem of applying a tight urisheath on a relaxed penis.

### SUMMARY OF THE INVENTION

It is therefore one object of the current invention to provide a relatively simple attachment member for a urine collecting system, that is easy to apply on a person with a small and/or relaxed penis, for example an elderly person's penis, and that does not unintentionally fall off.

In a first aspect, the present invention relates to an attachment member suitable for attaching a system to a penis; said attachment member comprising: at least one elastic film material having a part that is substantially cylindrically shaped; and at least two openings; the two openings being able to be expanded to a diameter larger than the diameter of the outer surface of the penis.

More specifically, the present invention solves the above mentioned object by providing an attachment member as mentioned in the introductory paragraph where the inner diameter of the rolled up portion of the substantially cylindrical portion is less than 45 mm when the elastic film material of the attachment member is not stretched and not rolled up. In this way an attachment member is provided which is easy to place on an inner portion of a penis and can then be unrolled in a direction away from the base of the penis. Due to the elasticity of the material and the diameter of the cylindrical portion, the attachment member forms a secure fit on the penis.

The value of 45 mm has been chosen to reflect the statistical diameters of the average penis diameter. In order to cover the most common penis diameters found in the adult male population, the relaxed inner diameter should be less than 45 mm so that an effective seal is provided when applied on the penis. In practise, a series of attachment members will be supplied in different diameters where the diameters will fall within the range of less than 45 mm. Of course there are penis diameters which will fall outside this range, but the commercially interesting range is less than 45 mm. However, in certain embodiments, the relaxed inner diameter could be less than 40 mm or less than 38 mm or less than 35 mm.

It should be noted that the substantially cylindrical portion is in one embodiment made from an elastic film material. The substantially cylindrical portion could furthermore be arranged such that the inner diameter of the substantially cylindrical portion can be stretched.

In a second aspect, the present invention relates to a system suitable for collecting urine from a male human being, the system comprising a urine container for collecting and/or storing urine during wear time; an attachment member for attaching the system to a penis, the attachment member comprising: at least one elastic film material having a part that is substantially cylindrically shaped; and at least two openings; the two openings being able to be expanded to a diameter larger than the diameter of the outer surface of the penis.

More specifically, the present invention relates to a system for collecting urine from a male human being said system comprising an attachment member as described above with respect to the first aspect of the present invention.

In a third aspect, the present invention relates to a method of attaching an attachment member, suitable for connecting a urine collecting system to a penis, to a penis, the method comprising the steps of: providing an attachment member comprising an elastic film material; said elastic film having a part that is substantially cylindrically shaped; positioning at least a part of the attachment member on the surface of the penis at an inwards position on the penis rod, and unrolling at least a part of the attachment member in the outwards direction of the penis away from the base of the penis.

In the context of the present invention, an “inwards position” on the penis is defined as a position between the symphysis and the glans penis, while an “outwards position” is on the glans penis.

More specifically the third aspect of the present invention relates to a method of attaching an attachment member to a penis comprising the steps of providing an attachment member comprising a substantially cylindrical portion which: comprises an elastic film material, has a first opening at a first end thereof and a second opening at the other end thereof, is arranged such that at least the glans penis of a penis could be arranged inside the substantially cylindrical portion with the first opening being further from the base of the penis than the second opening, and is at least partly rolled up to assume a ring like configuration in such a way that the first opening is located inside the rolled up portion and the second opening is located outside the rolled up portion, stretching the rolled up portion and the second opening such that the inner diameter of the rolled up portion and the diameter of the second opening are at least 1% greater than the inner diameter of the rolled up portion of the substantially cylindrical portion when the elastic film material of the substantially cylindrical portion is not stretched and not rolled up, positioning the attachment member on the outer surface of the penis rod at a position between the glans penis and the base of the penis; and unrolling at least a part of the attachment member in a direction along the penis and away from the base of the penis.

In another embodiment of the method, the method could further comprise the step of releasing the stretching force applied to the attachment member and allowing it to retract, after positioning the attachment member on the outer surface of the penis.

In yet a further embodiment, the method could further comprise the steps of determining the approximate outer diameter of the penis and providing an attachment member which is sized appropriately for the approximated outer diameter of the penis.

It should be noted that the above mentioned methods are not a methods of treatment by therapy since the above mentioned methods do not cure or stabilize the condition of the user of the system. Furthermore, the methods do not result in a significant health risk to the user if the methods are performed incorrectly. As such, non-trained medical staff can perform the method.

### DETAILED SUMMARY OF THE INVENTION

A first aspect of the present invention relates to an attachment member for attaching a system to a penis; said attachment member comprising: at least one elastic film material having a part that is substantially cylindrically shaped; and at least two openings; the two openings being able to be expanded to a diameter larger than the diameter of the outer surface of the penis.

In the context of the present invention the term “attachment member” shall be understood as a member adapted to contact and to be attached to a penis.

In the context of the present invention the term “elastic film” shall be understood as a thin sheet like material that via a force can be expanded and will contract when the force is removed. The sheet like material may in some cases essentially return to its original shape.

In the context of the present invention the term “substantially cylindrical shape” shall be understood as a shape formed like a pipe, a cylinder, a truncated cone, a diamond shape, a cylindrical shape with varying cross sectional diameter, an oval cylinder, combinations of the above or other cylindrically shaped structures with regular or irregular cross sectional geometries.

Since it is difficult to create a seal against the skin surface of a penis an elastic film material is used. It is important that the material is elastic enough to expand and/or to conform to the changing shape of the penis, but also designed to stay in place, which is why the size and elasticity of the substantially cylindrical shape is important.

In one embodiment of the first aspect of the present invention, the combined elasticity of the elastic film and the flexibility of the penis create a seal and/or a fastening of the attachment member to the penis.

In one embodiment of the attachment member, the substantially cylindrical portion of the attachment member is essentially liquid impermeable, at least in the areas which are in direct contact with urine during the wear-time of the attachment member.

In one embodiment of the first aspect of the present invention, the inner diameter of at least a part of the substantially cylindrically shaped elastic film is smaller than the outer diameter of the penis rod. In one embodiment of the first aspect of the present invention, the diameter of at least a part of the substantially cylindrically shaped elastic film is between 0.5 and 90 percent smaller than the diameter of the penis rod. In one embodiment of the first aspect of the present invention, the diameter of at least a part of the substantially cylindrically shaped elastic film is between 1 and 50 percent smaller than the diameter of the penis rod. In one embodiment of the first aspect of the present invention, the diameter of at least a part of the substantially cylindrically shaped elastic film is between 5 and 25 percent smaller than the diameter of the penis rod. In one embodiment of the first aspect of the present invention, the diameter of at least a part of the substantially cylindrically shaped elastic film is between 15 and 30 percent smaller than the diameter of the penis rod.

It should be noted, that for the sake of this specification, when reference is made to “the outer diameter of the penis”, then the diameter should be a penis diameter which can be found within approximately the 90^{th} percentile of the penis diameters of the world's adult male human being population. Therefore, “the diameter of the penis” should not be interpreted as a single value, but as a range of values. While such data is not readily available, the person skilled in the art will know how to approximate it. In case it is necessary, then “the diameter of the penis” can be restricted to a range of between 15 and 50 mm or between 15 and 45 mm or between 20 and 45 mm or between 20 and 40 mm or between 25 and 36 mm or between 32 and 44 mm. Furthermore, since the outer diameter of a male human being's penis changes over time, the outer diameter of the penis as mentioned in this specification should refer to the flaccid or minimum outer diameter of a human being's penis.

In one embodiment, the diameter of the two openings and the film material should be designed so that the two openings are expandable to a size larger than the outer diameter of the penis, but also designed so that their relaxed diameter is smaller than the outer diameter of the penis. In this way, it is possible to apply the attachment member onto the penis when the openings are expanded and then create a good seal when the openings are allowed to retract.

In one embodiment of the first aspect of the present invention, the diameters of the two openings are at least 14 mm. In one embodiment of the first aspect of the present invention the diameters of the two openings are at least 16 mm. In one embodiment of the first aspect of the present invention the diameters of the two openings are at least 18 mm. In one embodiment of the first aspect of the present invention the diameters of the two openings are at least 20 mm. In one embodiment of the first aspect of the present invention the diameters of the two openings are at least 22 mm.

In one embodiment of the first aspect of the present invention, the diameter of the second opening is greater than 20 mm when the second opening is not stretched. The value of 20 mm is again chosen based on a statistical range as a minimum value for this particular embodiment. In other embodiments, the diameter of the second opening could be greater than 22 mm or greater than 24 mm or greater than 26 mm.

It is important, when choosing the elasticity of the elastic film, that the force of attachment (both in relation to fastening and to discomfort feeling) and easiness to apply are considered. Consequently, the physical properties of the elastic film material are important.

In the context of the present invention, the term “elongation of the elastic film material” describes the elasticity of the material and is measured according to the standard test method ASTM D638 as the elongation at break (measured in percent).

In one embodiment of the first aspect of the present invention, the substantially cylindrically shaped elastic film material is able to be elongated between 100 and 2000 percent. In one embodiment of the first aspect of the present invention the substantially cylindrically shaped elastic film material is able to be elongated between 400 and 1900 percent. In one embodiment of the first aspect of the present invention the substantially cylindrically shaped elastic film material is able to be elongated between 600 and 1700 percent. In one embodiment of the first aspect of the present invention, the substantially cylindrically shaped elastic film material is able to be elongated between 1000 and 1500 percent.

In the context of the present invention, the term “modulus 300%” describes the stress (measured in MPa) required to elongate a sample of given dimensions 300%, measured according to the standard test method ASTM D638.

In one embodiment of the first aspect of the present invention, the substantially cylindrically shaped elastic film material has a modulus 300% of between 0.05 and 1.00. In one embodiment of the first aspect of the present invention the substantially cylindrically shaped elastic film material has a modulus 300% of between 0.07 and 0.60. In one embodiment of the first aspect of the present invention the substantially cylindrically shaped elastic film material has a modulus 300% of between 0.08 and 0.40. In one embodiment of the present invention, the substantially cylindrically shaped elastic film material has a modulus 300% of between 0.09 and 0.30.

In the context of the present invention, the term “modulus 100%” describes the stress (measured in MPa) required to elongate a sample of given dimensions 100%, measured according to the standard test method ASTM D638.

In one embodiment of the first aspect of the present invention, the substantially cylindrically shaped elastic film material has a modulus 100% of between 0.02 and 0.50. In one embodiment of the first aspect of the present invention the substantially cylindrically shaped elastic film material has a modulus 100% of between 0.03 and 0.30. In one embodiment of the first aspect of the present invention the substantially cylindrically shaped elastic film material has a modulus 100% of between 0.04 and 0.20. In one embodiment of the first aspect of the present invention, the substantially cylindrically shaped elastic film material has a modulus 100% of between 0.05 and 0.15.

It should be noted that both the type of material used and the thickness of the material used for the elastic film material has an effect on the force of elongation. Consequently, the choice of material and the choice of wall thickness are both important when selecting material for the elastic film.

In one embodiment of the first aspect of the present invention, the elastic film material is selected from a group comprising thermoplastic elastomers and silicones. In one embodiment of the first aspect of the present invention the elastic film material has a shore OO hardness between 10 and 90, or between 15 and 80. In one embodiment of the first aspect of the present invention the elastic film material has a shore OO hardness between 20 and 70. In one embodiment of the first aspect of the present invention the elastic film material has a shore OO hardness between 25 and 65.

In one embodiment of the first aspect of the present invention, the substantially cylindrically shaped elastic film has in its unrolled and relaxed and non-stretched configuration a second opening adapted to receive the penis, the inner diameter of said second opening being identical to or smaller than the outer diameter of the penis. In one embodiment of the first aspect of the present invention the second opening of the attachment member has a relaxed diameter that is between 1 and 90 percent smaller than the smallest diameter size of the penis. In one embodiment of the first aspect of the present invention the second opening of the attachment member has a diameter that is between 5 and 40 percent smaller than the smallest diameter size of the penis. In one embodiment of the first aspect of the present invention the second opening of the attachment member has a diameter that is between 10 and 25 percent smaller than the smallest diameter size of the penis.

In one embodiment of the first aspect of the present invention, the attachment member is adapted to be positioned at an inwards position on the penis. In one embodiment of the first aspect of the present invention the attachment member is adapted to be unrolled outwardly from the inwards position on the penis in a direction away from the base of the penis.

In one embodiment of the first aspect of the present invention, the substantially cylindrically shaped elastic film has an area/portion adapted to receive and seal against a penis during wear time, said area/portion having a diameter identical to or smaller than the diameter of the penis. In one embodiment of the first aspect of the present invention the area/portion of the attachment member adapted to receive and seal against the penis has a diameter that is between 1 and 80 percent smaller than the diameter of the penis in an inwards position. In one embodiment of the first aspect of the present invention the area/portion of the attachment member adapted to receive and seal against the penis has a diameter that is between 3 and 40 percent smaller than the diameter of the penis in an inwards position. In one embodiment of the first aspect of the present invention the area/portion of the attachment member adapted to receive and seal against the penis has a diameter that is between 6 and 20 percent smaller than the diameter of the penis in an inwards position.

In order to apply the attachment member on the penis, the rolled up portion and the second opening could be stretched such that the inner diameter of the rolled up portion and the diameter of the second opening are at least 10% greater than the inner diameter of the rolled up portion of the substantially cylindrical portion when the elastic film material of the substantially cylindrical portion is not stretched and not rolled up. In other embodiments, the diameter could be stretched at least 20%, 30%, 40%, 50%, 60%, 70%, 80%, 90% or 100%. The greater the amount that diameter is stretched, the easier it is to mount the attachment member on the penis.

In one embodiment of the first aspect of the present invention, the attachment member comprises a fastening element for more securely attaching the attachment member to the surface of the skin of a penis.

In the context of the present invention the term “fastening element” shall be understood as any form, shape or area of the attachment member, supporting the attachment of the member to a penis, and supporting the attachment member in staying in place during the wear-time of the urine collecting system.

In the context of the present invention the term “wear-time” shall be understood as the time period the attachment member is intended to stay on the penis. Such time may vary from a few minutes up to a number of days, for example 2, depending on the condition of the patient.

In one embodiment of the first aspect of the present invention the fastening element comprises an adhesive for attaching the member to the surface of the skin of a penis. In one embodiment of the first aspect of the present invention at least an area of the elastic film material comprises an adhesive for attachment to the surface of the skin on a penis. In one embodiment of the first aspect of the present invention the adhesive is a non absorbent adhesive. Such adhesives may be any adhesive, for example an acrylic adhesive; a polyurethane adhesive, a silicone adhesive or the like. In one embodiment of the first aspect of the present invention, the adhesive for attaching the attachment member to the penis is a silicone adhesive.

Adhesives may damage the skin when applied several times or when worn for a prolonged period of time. In order to reduce the stress to the skin on the penis, it may be advantageous to apply the adhesive in a pattern.

In one embodiment of the first aspect of the present invention the attachment member comprises an adhesive for attaching the member to the skin; said adhesive being presented in a pattern which covers an area. In one embodiment of the first aspect of the present invention the adhesive pattern covers 10 to 90 percent of the total adhesive area. In one embodiment of the first aspect of the present invention the adhesive pattern covers 25 to 80 percent of the adhesive area. In one embodiment of the first aspect of the present invention the adhesive pattern covers 40 to 70 percent of the adhesive area.

In order to ease the application of an urisheath, prior to application, the urisheath is put into a pre-rolled form which is then unrolled during application of the system. Furthermore due to the form of the urisheath according to the current invention, the direction of which the urisheath is unrolled relative to the penis can be changed with respect to the current direction of unrolling urisheaths in order to ease the unrolling process.

In one embodiment of the first aspect of the present invention, the substantially cylindrical portion of the attachment member is adapted to be rolled up prior to being positioned on a penis. In this embodiment the attachment member is provided to the user in an unrolled form and then rolled up prior to application. In another embodiment, the attachment member is provided in an already rolled up form.

In one embodiment of the first aspect of the present invention, the substantially cylindrical portion of the attachment member is adapted to be unrolled after being positioned in an inwards position on the penis. In one embodiment of the first aspect of the present invention the attachment member is adapted to be rolled up prior to being positioned on the penis and adapted to be unrolled after being positioned in an inwards position on the penis.

In one embodiment of the first aspect of the present invention, the substantially cylindrical portion of the attachment member is adapted to be unrolled on a penis in an outwards direction, i.e. away from the body of the user. In one embodiment of the first aspect of the present invention the attachment member is adapted to be unrolled a distance which extends past the glans penis.

In one embodiment of the first aspect of the present invention, the substantially cylindrical portion of the attachment member is adapted to be positioned at an inwards position on a penis prior to unrolling the member. In one embodiment of the first aspect of the present invention at least a part of the attachment member remains at an inwards position after the attachment member has been unrolled.

Depending on how the attachment member is attached to the penis, it may be important that the length of the urisheath is longer than the length of the penis in order to ensure that the connection of the system for collecting urine is further from the body of the user than the glans penis.

In one embodiment of the first aspect of the present invention, the attachment member comprises a connecting element.

In the context of the present invention, a “connecting element” shall be understood as an area/portion/element of the attachment member that is used for connecting the attachment member to a connecting member of a urine collecting system. It may be of any form, material or shape which is suitable for this purpose.

In one embodiment of the first aspect of the present invention, the attachment member comprises a connecting element, said connecting element adapted to be positioned further from the base of the penis than the glans penis when attached to the penis.

In one embodiment of the first aspect of the present invention, the attachment member is between 1 and 50 cm long. In one embodiment of the first aspect of the present invention the attachment member is between 2 and 40 cm long. In one embodiment of the first aspect of the present invention the attachment member is between 4 and 30 cm long. In one embodiment of the first aspect of the present invention the attachment member is between 8 and 24 cm long. In one embodiment of the first aspect of the present invention the attachment member is between 11 and 19 cm long.

It may be difficult to apply the attachment member correctly on a penis due to the nature of the penis and due to the material characteristics of the elastic film. Therefore it may be desirable to use an applicator to apply the attachment member.

The attachment member could therefore be provided in a kit of parts comprising an attachment member as provided above and an applicator which is arranged to stretch the rolled up portion of the substantially cylindrical portion such that the inner diameter of the rolled up portion is increased by at least 10% when compared to its non-stretched inner diameter. In different embodiments, the applicator could be arranged to increase the inner diameter at least 20%, 30%, 40%, 50%, 60%, 70%, 80%, 90% or 100%.

In the context of the present invention, an “applicator” shall be understood as a physical form and/or shape and/or mechanism adapted to support at least the positioning of at least a part of the attachment member in an inwards position on a penis. The applicator may be a separate element or it may be integrated into the attachment member.

In one embodiment of the first aspect of the present invention the attachment member comprises an applicator for positioning the attachment member on the skin of a penis in an inwards position. In one embodiment of the first aspect of the present invention the applicator is adapted to expand the diameter of the substantially cylindrical shape of the elastic film to a size larger than the outer diameter of the penis.

In one embodiment of the first aspect of the present invention the applicator is adapted to: expand the diameter of the substantially cylindrical shape of the elastic film to a size larger than the outer diameter of the penis; and to support the positioning of at least a part of the attachment member on the penis in an inwards position.

In one embodiment of the first aspect of the present invention the applicator is adapted to expand the diameter of the substantially cylindrical shape of the elastic film to a size larger than the outer diameter of the penis; to support the positioning of at least a part of the attachment member on the penis in an inwards position; and to support the unrolling process of the attachment member in an outwards direction.

In one embodiment of the first aspect of the present invention, the applicator is integrally formed with the attachment member.

In one embodiment of the first aspect of the present invention, the applicator comprises an elastomeric material. In one embodiment of the first aspect of the present invention the elastomeric material is selected from a group comprising thermoplastic elastomers and silicones. In one embodiment of the first aspect of the present invention the elastomeric material has a shore D hardness between 2 and 80, or between 5 and 60. In one embodiment of the first aspect of the present invention the elastomeric material has a shore D hardness between 10 and 35.

In one embodiment of the first aspect of the present invention, the applicator is made of a polymeric material. In one embodiment of the first aspect of the present invention the applicator comprises a thermoplastic material. In one embodiment of the first aspect of the present invention the applicator comprises a biodegradable polymeric material.

In one embodiment of the first aspect of the present invention, the applicator comprises a ring having a circular or oval shape. In one embodiment of the first aspect of the present invention, the ring has an inner diameter larger than the outer diameter of the penis.

In one embodiment of the first aspect of the present invention, the applicator is disconnected from the attachment member during application of the attachment member to a penis.

In one embodiment of the first aspect of the present invention, the applicator is adapted to be removed from the attachment member after the attachment member has been applied to a person.

In one embodiment of the first aspect of the present invention, the attachment member is pre-rolled around the applicator such that the applicator is arranged inside the rolled up portion of the substantially cylindrical portion.

In order to ease the unrolling process, the applicator should be designed to enable a simple and even unrolling process.

In one embodiment of the first aspect of the present invention, the applicator is easily rolled around its centre during the unrolling process.

In one embodiment of the first aspect of the present invention, at least a portion of the elastic film material has in its relaxed position a diameter equal to or smaller than the diameter of the penis it is designed to be attached to, but when rolled around the applicator the diameter of the portion of the elastic film is expanded to a diameter larger than the diameter of the penis.

In one embodiment of the first aspect of the present invention, the applicator is formed from a shaped stick.

In the context of the present invention, a “shaped stick” shall be understood as any physical form and/or shape and/or mechanism wherein the form/shape/mechanism is oblong.

In one embodiment of the first aspect of the present invention, the shaped stick is made with a cylindrical cross section. In one embodiment of the first aspect of the present invention, the shaped stick is bent to form a circular or oval shape. In one embodiment of the first aspect of the present invention, the shaped stick is bent to form a circular shape, wherein the inner diameter of the circular shape is larger than the diameter of the second opening of the attachment member. In one embodiment of the first aspect of the present invention, the shaped stick is bent to form a circular shape, wherein the inner diameter of the circular shape is at least 1 mm larger than the diameter of the second opening of the attachment member.

In one embodiment of the first aspect of the present invention, the shaped stick has a connect form and a receptor; wherein the receptor is adapted to receive the connect form.

In the context of the present invention, a “receptor” shall be understood as a physical form and/or shape and/or mechanism on the applicator adapted to receive a connect form.

In the context of the present invention, a “connect form” shall be understood as a physical form and/or shape and/or mechanism on the applicator adapted to engage with a receptor.

In one embodiment of the first aspect of the present invention, the shaped stick has a connect form and a receptor; wherein the receptor is adapted to receive the connect form. In one embodiment of the first aspect of the present invention, the receptor is adapted to allow the connect form to stay connected to the receptor. In one embodiment of the first aspect of the present invention, the shaped stick forms a circular shape when the receptor and the connect form are connected.

In one embodiment of the first aspect of the present invention, the receptor and the connect form are formed at respective ends of the shaped stick.

The shaped stick may be a massive or hollow stick. In one embodiment of the first aspect of the present invention, the shaped stick is hollow. In one embodiment of the first aspect of the present invention, the shaped stick is massive.

In one embodiment of the first aspect of the present invention, the attachment member comprises a substantially ring formed applicator, wherein the inner diameter of the applicator is larger than the diameter of the penis.

In one embodiment of the first aspect of the present invention, the applicator comprises a shaped stick.

In one embodiment of the first aspect of the present invention, at least a part of the attachment member is pre-rolled around a shaped stick and adapted to be uncoiled in an outwards direction on the penis.

As a soft thermoplastic elastomer has a tendency to creep over longer periods of time if stored in a stretched configuration, it may be desirable to allow the attachment member to during shelf life be stored in a relatively relaxed position and immediately prior to application on a penis be expanded.

In one embodiment of the first aspect of the present invention, the applicator has two configurations: a first configuration wherein the applicator has a first diameter; and a second configuration wherein the applicator has a second diameter; said second diameter being at least 20% larger than the first diameter. In other embodiments, the second diameter could be at least 30%, 40%, 50%, 60%, 70%, 80%, 90% or 100% larger than the first diameter.

In one embodiment of the first aspect of the present invention, the applicator is adapted to expand the diameter of at least a portion of the elastic film by at least 1 mm; preferably the applicator is adapted to expand the diameter of at least a portion of the elastic film by at least 2 mm; and most preferably the applicator is adapted to expand the diameter of at least a portion of the elastic film between 2 and 10 mm.

In one embodiment of the first aspect of the present invention, the shaped stick is in the form of a spring.

Such a spring has some advantages in use. The spring may, after it has been bent to a circular shape, be compressed. The compressed spring, comprising the rolled-up portion of the cylindrically shaped elastic film, may be stored in the compressed form in a packaging. Then when removing the spring and attachment member from the packaging, the spring automatically expands and enlarges the diameter of the rolled-up portion of the attachment member.

In one embodiment of the first aspect of the present invention, the spring is compressible. In one embodiment of the first aspect of the present invention, the spring is compressible to between 20 and 80% of its relaxed length. In one embodiment of the first aspect of the present invention, the spring is compressible to between 30 and 70% of its relaxed length.

The material and thickness of the spring dictate the stress the spring will impact on the elastic film.

In one embodiment of the first aspect of the present invention, the spring is made of a metal. In one embodiment of the first aspect of the present invention, the spring is made of a plastic material.

In one embodiment of the first aspect of the present invention, the spring is made in a spiral form from a wire thickness of between 0.1 and 1.0 mm. In one embodiment of the first aspect of the present invention, the spring is made of a wire thickness of between 0.2 and 0.5 mm.

In one embodiment of the first aspect of the present invention, the applicator comprises at least two shaped sticks; said shaped sticks adapted to engage with each other and adapted to expand the diameter of the applicator.

In one embodiment of the first aspect of the present invention, the substantially cylindrically shaped elastic film has a first opening adapted to allow fluid communication between the penis and an external container.

In one embodiment of the first aspect of the present invention, the substantially cylindrically shaped elastic film has a first opening; said first opening is capable of being expanded to a diameter larger than the diameter of the penis. In one embodiment of the first aspect of the present invention, the first opening of the substantially cylindrically shaped elastic film has a diameter that is larger than the diameter of the penis. This will reduce the force which tries to pull the attachment member off the penis during wear-time.

In one embodiment of the first aspect of the present invention, the substantially cylindrically shaped elastic film has a first opening adapted to allow fluid communication between the penis and the external container; said first opening is capable of being expanded to a diameter larger than the diameter of the penis.

In one embodiment of the first aspect of the present invention, the entire attachment member is adapted to be positioned at an inwards position on a penis and adapted to be unrolled outwardly; said unrolling allowing the second opening to remain in an inwards position and said first opening to obtain a new position, the new position being further from the base of the penis than the glans penis.

The thickness of the elastic film is important with respect to avoiding unintended cracks and/or holes in the film, but also in relation to the comfort of the user during wear time.

In one embodiment of the first aspect of the present invention the substantially cylindrically shaped elastic film has a thickness of between 0.01 and 2 mm. In one embodiment of the first aspect of the present invention the thickness of the cylindrically shaped elastic film is between 0.04 and 0.8 mm. In one embodiment of the first aspect of the present invention the thickness of the cylindrically shaped elastic film is between 0.08 and 0.4 mm. In one embodiment of the first aspect of the present invention the thickness of the cylindrically shaped elastic film is between 0.1 and 0.3 mm. In one embodiment of the first aspect of the present invention the thickness of the cylindrically shaped elastic film is between 0.15 and 0.25 mm.

In order to ease the unrolling process, it may be necessary to support the unrolling process with an unrolling supportive element. Such unrolling supportive element(s) may also reduce the need to touch the penis during application of the member. This may be particularly desirable if the member is applied by another person than the human being intended to wear the attachment member.

In the context of the present invention, an “unrolling supportive element” shall be understood as a physical form and/or shape and/or mechanism adapted to support the unrolling process of an attachment member in an outwards direction on a penis.

In one embodiment of the first aspect of the present invention, the attachment member comprises an unrolling supportive element. In one embodiment of the first aspect of the present invention, the unrolling supportive element comprises at least one strip. In one embodiment, the unrolling supportive element comprises at least two strips.

In order to ease the unrolling process, it may be advantageous to reduce the adherence of the elastic film material to itself. This may be achieved by introducing a non-adherent layer (which then also needs to be elastic/expandable) or by altering the surface properties of the elastic film material.

In one embodiment of the first aspect of the present invention, the elastic film material is non-adherent to itself. In one embodiment of the first aspect of the present invention, the elastic film material is coated with talc.

Additionally, it may be painful or uncomfortable or irritating if crotch hairs entangle with the attachment member during the unrolling process. It may consequently be convenient to shield crotch hairs from the attachment member with a “shield element”.

In the context of the present invention, a “shield element” shall be understood as a physical form and/or shape and/or mechanism adapted to shield at least a portion of the user's crotch hairs during the uncoiling process. Such a shield element may be in the form of a separate element or it may be integrally formed with the attachment member and/or applicator.

In one embodiment of the first aspect of the present invention, the attachment member comprises a shield element. In one embodiment of the first aspect of the present invention, the applicator comprises a shield element. Furthermore, in one embodiment a kit of parts could be provided which comprises an attachment member as described above or a kit of parts as described above where the kit could further comprise a shield element arranged at the second opening of the substantially cylindrical portion of the attachment member. In one embodiment, said shield element could comprise a flange having an outer diameter which is at least 2 cm greater than the inner diameter of the rolled up portion of the substantially cylindrical portion when the rolled up portion is not rolled up and not stretched.

In one embodiment of the first aspect of the present invention, the shield element comprises a flange portion. The flange portion may be used to shield the crotch hairs, but may also used as an unrolling supportive element as the person applying the attachment member may use the flange as a grip during the unrolling process, thereby ensuring that a portion of the attachment member stays in an inwards position on the penis.

In one embodiment of the first aspect of the present invention the flange portion is integrally formed with the attachment member.

In one embodiment of the first aspect of the present invention the outer diameter of the flange portion is between 5.0 and 25.0 cm. In one embodiment of the first aspect of the present invention the diameter of the flange portion is between 6.0 and 20 cm. In one embodiment of the first aspect of the present invention the diameter of the flange portion is between 7.0 and 18.0 cm. In one embodiment of the first aspect of the present invention the diameter of the flange portion is between 8.0 and 16.0 cm. In one embodiment of the first aspect of the present invention the diameter of the flange portion is between 6.5 and 12.5 cm. In one embodiment of the first aspect of the present invention the diameter of the flange portion is between 5.9 and 10.5 cm.

In one embodiment of the first aspect of the present invention, the shield element comprises a truncated cone portion.

In one embodiment of the first aspect of the present invention, the attachment member comprises: an applicator for applying the attachment member in an inwards position on the penis prior to unrolling at least a part of the attachment member; and a connecting element for connecting the attachment member to a system.

In one embodiment of the first aspect of the present invention, the attachment member comprises: at least one elastic film material having a part that is substantially cylindrically shaped, the substantially cylindrically shaped elastic film having an area adapted to receive and seal against a penis, said area having a diameter that is smaller than the diameter of the penis; at least two openings; the two openings being able to be expanded to a diameter larger than the diameter of the outer surface of the penis; and a substantially ring formed applicator, wherein the inner diameter of the applicator is larger than the diameter of the penis.

In one embodiment of the first aspect of the present invention, the substantially cylindrically shaped elastic film has an area adapted to receive and seal against a penis, said area having a diameter that is smaller than the diameter of the penis.

In one embodiment of the first aspect of the present invention, the attachment member is adapted to be expanded to a diameter larger than the diameter of the penis.

In one embodiment of the first aspect of the present invention, the elastic film material, having a part that is cylindrically shaped, is characterised in that the diameter of the shape varies along the length of the substantially cylindrical shape. In one embodiment of the second aspect of the present invention the diameter varies according to the variations of a penis. In one embodiment of the second aspect of the present invention the varying cylindrical shape comprises a smaller diameter adapted to fit the inwards position of a penis; and a larger diameter adapted to fit to the glans penis. In one embodiment of the first aspect of the present invention the varying cylindrical shape comprises a shape similar to a penis shape. In one embodiment of the first aspect of the present invention, the varying cylindrical shape takes the form of a wave, with periodically increasing and decreasing diameter.

The skin on a human being wearing an urisheath for a prolonged period of time may be stressed. Such stress may result in skin irritation and eventually in wounds. Consequently it may be desirable to add ingredients to the elastic film material which care for the skin on the penis. Such skin caring ingredients may be antibacterial, antifungal and natural ingredients such as aloe vera.

In one embodiment of the first aspect of the present invention, the elastic film material comprises a skin caring ingredient. In one embodiment of the first aspect of the present invention, the elastic film material comprises an antibacterial material. In one embodiment of the first aspect of the present invention, the elastic film material comprises a silver containing compound as antibacterial ingredient. In one embodiment of the first aspect of the present invention, the elastic film material comprises a silver zirconium phosphate compound as the antibacterial ingredient.

In one embodiment of the first aspect of the present invention, the elastic film material is coated with a blend of talc and a skin caring ingredient. In one embodiment of the first aspect of the present invention, the elastic film material is coated with a blend of talc and a silver zirconium phosphate compound. In one embodiment of the first aspect of the present invention, the elastic film material is coated with a blend of talc and Alphasan 2000 from Milliken Chemical.

A second aspect of the present invention relates to a system for collecting urine from a male human being, the system comprising: a urine container for collecting and/or storing urine during wear time; an attachment member for attaching the system to a penis, the member comprising: at least one elastic film material having a part that is substantially cylindrically shaped; and at least two openings; the two openings being able to be expanded to a diameter larger than the diameter of the outer surface of the penis.

In the context of the present invention the term “urine container” shall be understood as any type of encapsulated volume, for example a bag or rigid container.

One embodiment of the second aspect of the present invention relates to a system for collecting urine, the system comprising: a connecting member adapted to connect the attachment member to the system.

In the context of the present invention the term “connecting member” shall be understood as a member adapted to bring the interior of the attachment member into fluid communication with the interior of the urine container.

In one embodiment of the second aspect of the present invention the connecting member and a connecting element of the attachment member are adapted to engage with each other. In one embodiment of the second aspect of the present invention the connecting member and a connecting element of the attachment member are adapted to engage with each other and create a fluid tight seal.

In one embodiment of the second aspect of the present invention, the connecting element is adapted to reversibly connect to and disconnect from the urine collecting system. In one embodiment of the second aspect of the present invention, the connecting member is adapted to reversibly connect to and disconnect from the urine collecting system.

In one embodiment of the second aspect of the present invention, the connecting element and/or connecting member comprises a locking mechanism for locking the position of the connecting element relative to the collecting member and/or urine collecting system.

In the context of the present invention, a “locking mechanism” shall be understood as a physical form and/or shape and/or mechanism adapted to support the engagement of the connecting element to the connecting member and/or system for collecting urine.

In one embodiment of the second aspect of the present invention, the urine collecting system comprises a locking mechanism. In one embodiment of the second aspect of the present invention the locking mechanism is adapted to lock and unlock in order to replace at least a part of the system.

One embodiment of the second aspect of the present invention relates to a system for collecting urine, the system comprising an adhesive, said adhesive supporting the connection and seal between the connecting element and the connecting member. The position and/or area of the adhesive may vary depending on the design of the connecting element and/or the design of the connecting member.

In one embodiment of the second aspect of the present invention, the adhesive covers at least an area of the outer surface of the connecting member. In one embodiment of the second aspect of the present invention, the adhesive covers at least an area of the inner surface of the connecting member. In one embodiment of the second aspect of the present invention, the adhesive is covered by a non touch film.

One embodiment of the second aspect of the present invention relates to a system for collecting urine, the system comprising a connecting member; said connecting member adapted to expand the inner diameter of the connecting element of the attachment member.

In one embodiment of the second aspect of the present invention the urine container is a urine collecting bag. In one embodiment of the second aspect of the present invention the bag may be drained from liquid. In one embodiment of the second aspect of the present invention the bag comprises attachment elements for attaching the bag to a person and/or to a bed.

Some human beings only have a need for a small urine collecting capacity. In these cases the urine container may be designed to be very small and discrete.

In one embodiment of the second aspect of the present invention, the attachment member and the urine container are integrally formed.

In one embodiment of the second aspect of the present invention, the connecting member and the urine container are integrally formed.

In one embodiment of the second aspect of the present invention, the urine container is adapted to be encapsulated within the interior of the attachment member.

In one embodiment of the second aspect of the present invention, the urine container comprises a one way valve allowing liquid to enter the container, but not to leave the container.

In one embodiment of the second aspect of the present invention, the urine container comprises a reservoir.

In one embodiment of the second aspect of the present invention the reservoir comprises an absorbent fibrous material. In one embodiment of the second aspect of the present invention the reservoir comprises a liquid wicking fibrous material. In one embodiment of the second aspect of the present invention the reservoir comprises a fibre blend having both liquid wicking and liquid absorbent characteristics.

In one embodiment of the second aspect of the present invention the reservoir comprises an absorbent material in any shape. In one embodiment of the second aspect of the present invention the reservoir comprises absorbent particles.

In one embodiment of the second aspect of the present invention, the urine collecting system comprises an applicator for applying the attachment member on the skin of a penis in an inwards position. In one embodiment of the second aspect of the present invention the applicator is adapted to expand the diameter of the substantially cylindrical shape of the elastic film to a size larger than the outer diameter of the penis.

In one embodiment of the second aspect of the present invention the applicator is adapted to: expand the diameter of the substantially cylindrical shape of the elastic film to a size larger than the outer diameter of the penis; and to support the positioning of at least a part of the attachment member on the penis in an inwards position.

In one embodiment of the second aspect of the present invention the applicator is adapted to: expand the diameter of the substantially cylindrical shape of the elastic film to a size larger than the outer diameter of the penis; to support the positioning of at least a part of the attachment member on the penis in an inwards position; and to support the unrolling process of the attachment member in an outwards direction.

In one embodiment of the second aspect of the present invention, the system for collecting urine comprises a shield element.

In one embodiment of the second aspect of the present invention, the applicator comprises a shield element.

In one embodiment of the second aspect of the present invention, the shield element comprises a flange portion.

In one embodiment of the second aspect of the present invention, the system for collecting urine from a male human being comprises a universal connector for connecting the attachment member to the urine container. In one embodiment of the second aspect of the present invention the universal connector is adapted to reversibly connect to and disconnect from the urine collecting system.

In one embodiment of the second aspect of the present invention the universal connector is in the form of a separate element.

In the context of the present invention, a “universal connector” shall be understood as an integrated element of the attachment member or a separate element that is adapted to fit the attachment member to any standard urine collecting system. It may be of any form, material or shape.

The third aspect of the present invention relates to a method of attaching an attachment member for connecting a urine collecting system to a penis, the method comprising the steps of: providing an attachment member comprising an elastic film material; said elastic film having a part that is substantially cylindrically shaped, positioning at least a part of the attachment member on the surface of the penis in an inwards position; and unrolling at least a part of the attachment member in the outwards direction of the penis away from the base of the penis.

In one embodiment of the third aspect of the present invention, the method comprises the step of connecting the attachment member to a urine container, allowing the penis to be in fluid communication with the interior of the urine container. It is preferred, that the method comprises the step of locking the connection between the attachment member and the urine container.

In one embodiment of the third aspect of the present invention, the method comprises the step of adhering the attachment member to the skin of the penis.

In one embodiment of the third aspect of the present invention, the method comprises the step of creating a fluid tight connection between the attachment member and the surface of a penis.

In one embodiment of the third aspect of the present invention, the step of positioning at least a part of the attachment member in an inwards position on penis is achieved via the use of an applicator.

In one embodiment of the third aspect of the present invention, the step of positioning at least a part of the attachment member in an inwards position on penis and/or the step of unrolling at least a part of the attachment member in the outwards direction of the penis are achieved via the use of an applicator.

In one embodiment of the third aspect of the present invention, the method for applying an attachment member comprises the step of rolling the attachment member prior to application on the penis.

In one embodiment of the third aspect of the present invention, the method for applying an attachment member comprises the step of rolling the attachment member around an applicator prior to applying the attachment member on the penis.

In one embodiment of the third aspect of the present invention, the method for applying an attachment member comprises the step of removing the applicator from the attachment member after the step of unrolling at least a part of the attachment member.

In one embodiment of the third aspect of the present invention, the step of unrolling at least a part of the attachment member in an outwards direction of the penis is supported by an unrolling supportive element.

In one embodiment of the third aspect of the present invention, the approximate outer diameter of the penis is determined by a penis diameter measurement tool in order to choose the correct size attachment member.

In one embodiment of the third aspect of the present invention, the method comprises the step of closing a first opening of the attachment member.

In a preferred embodiment of the third aspect of the present invention, the method for applying an attachment member comprises the steps of: providing an attachment member comprising a cylindrically shaped elastic film and having two openings comprising the steps of; providing an applicator; pre-rolling at least a part of the attachment member around the applicator; positioning the attachment member on the outer surface of the penis in an inwards position; and unrolling at least a part of the attachment member in the outwards direction of the penis.

It will be appreciated that any combination of features and elements of the embodiments of the first, second and third aspects disclosed above may be combined in any suitable manner.

### BRIEF DESCRIPTION OF THE DRAWINGS

FIGS. 1a-1d disclose schematic cross sectional views of an attachment member for attaching a system to a penis according to the present invention. The figures show the attachment member in different configurations.

FIGS. 2 and 3 show schematic views of two different embodiments of an attachment member according to the current invention.

FIGS. 4a to 4c disclose schematic cross sectional views of how an attachment member may be applied on a penis according to one example of the present invention.

FIG. 5a discloses a schematic cross sectional view of an unrolled attachment member for attaching a system to a penis according to the present invention.

FIG. 5b discloses a schematic cross sectional view of an unrolled attachment member for attaching a system to a penis according to the present invention wherein the member is applied on a penis.

FIGS. 6a to 6c disclose schematic cross sectional views of an attachment member according to the present invention, the attachment member comprising an applicator.

FIGS. 7-8 disclose schematic cross sectional views of urine collecting systems according to the present invention.

FIGS. 9a-9c disclose schematic views of an applicator in the form of a shaped stick.

FIGS. 10a-10f disclose schematic views of an applicator in the form of two U-formed shaped sticks fitted together and forming one approximately circular bent stick applicator.

FIGS. 11a-11b disclose different views of a shield element.

FIGS. 12a-12e disclose views of an “external” applicator in the form of a separate element.

FIG. 13 discloses a cross sectional view of an applicator in the form of a separate element, wherein the applicator comprise a shield element

FIGS. 14a-b disclose a cross sectional view of an attachment member comprising a shield element.

FIG. 15 discloses a cross sectional view of an attachment member comprising a shield element and an unrolling supportive element.

FIG. 16 discloses a cross sectional view of an attachment member wherein the essentially cylindrically shaped elastic film has a varying inner diameter.

FIG. 17a-17c and FIGS. 18a-18c disclose cross sectional views of an attachment member and two different embodiments of a connecting member.

### DETAILED DESCRIPTION OF THE DRAWINGS

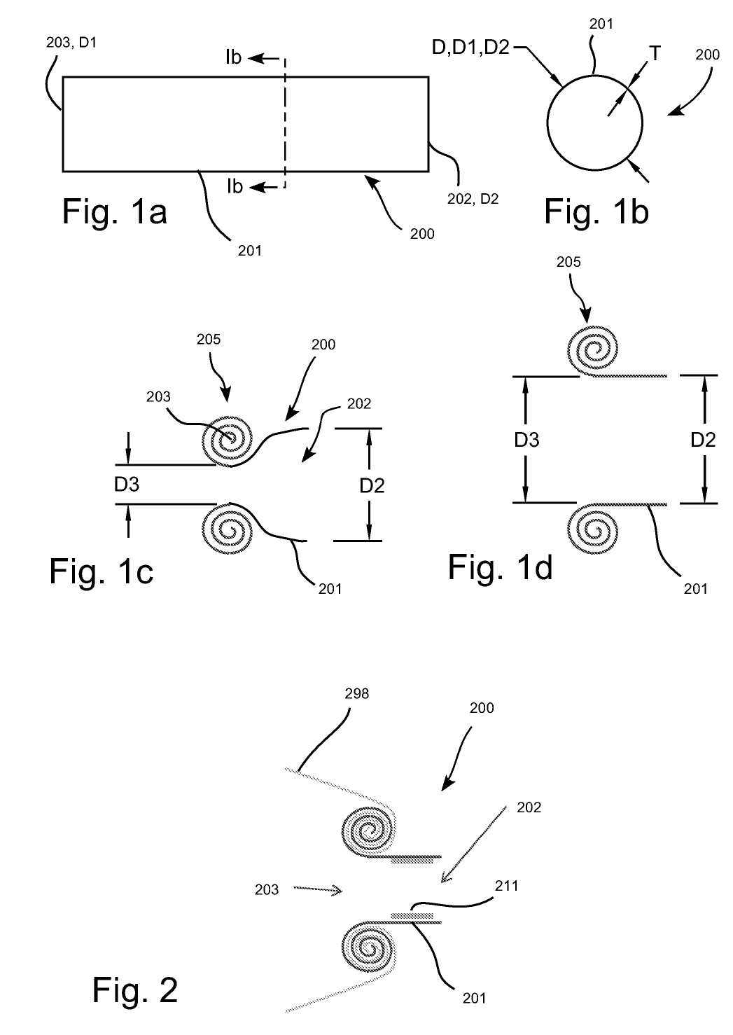

FIG. 1a shows a side view of a substantially cylindrical attachment member 200 formed from an elastic film material 201. FIG. 1b shows a cross section view of the attachment member 200 as defined by the line Ib-Ib in FIG. 1a. In general, the attachment member of this embodiment has a constant circular cross section with a diameter D and a wall thickness T. For the sake of this specification, the attachment member 200 is defined as having a first opening 203 at one end of the attachment member and a second opening 202 at the other end of the attachment member. In this embodiment, the inner diameter D1 of the first opening and the inner diameter of the second opening D2 are equal.

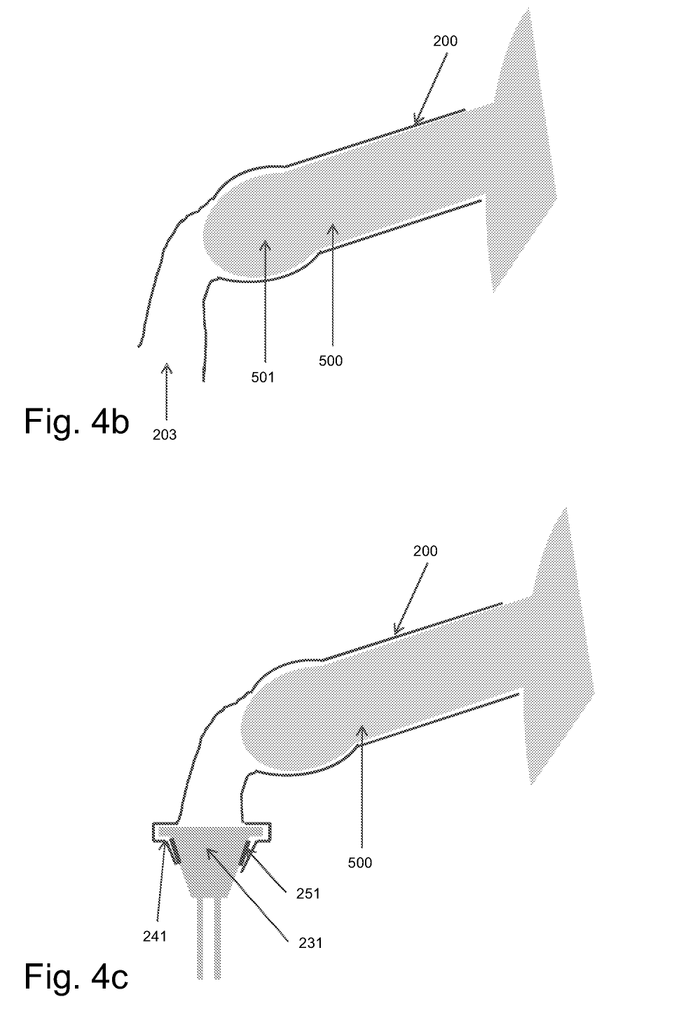

The attachment member is adapted to receive a penis and allow the elastic film to seal against the surface of the penis. The first and second openings 203,202 are arranged such that when the attachment member 200 is applied on a penis (see for example FIG. 4a) the first opening 203 is arranged further from the base of the penis than the second opening 202. In this way, the second opening is arranged close to the base of the penis when applied to the penis. The area of the attachment member near the first opening 203 is adapted as a connecting element which is suitable to receive a connecting member and to be fastened to the connecting member (see FIG. 4c).

It should be noted that in most cases, the attachment member will be longer than the penis length, see for example FIG. 4c due to the nature of typical connecting elements. However, it should be clear to the person skilled in the art, that if a connecting member were to be provided which was larger in diameter than the penis, then the attachment member could be shorter than the length of the penis.

FIG. 1c shows the attachment member of FIGS. 1a and 1b when it has been rolled up in a manner similar to a condom, but not stretched. However, in contrast to a condom, the rolled up attachment member as shown in figure is is open on both ends. As can be seen from FIG. 2, the diameter of the second opening D2 is still the same as before since it is not stretched, however the inner diameter D3 of the rolled up portion 205 of the attachment member is smaller than the diameter D1 and D2.

FIG. 1d shows the attachment member when it has been stretched. As can be seen from the figure both the inner diameter D3 of the rolled up portion 205 and the diameter D2 of the second opening 202 have been increased with respect to their original non-stretched diameters.

FIG. 2 discloses a schematic cross sectional view of another example of an attachment member 200 similar to FIG. 1c, but in FIG. 2 the attachment member comprises an adhesive 211 on the inner surface of the substantially cylindrical portion at the second opening 202 for adhesively attaching the attachment member to the surface of a penis. Furthermore the attachment member 200 comprises an unrolling supportive element 298 in the form of two strips, which, when pulled, will unroll the pre-rolled attachment member.

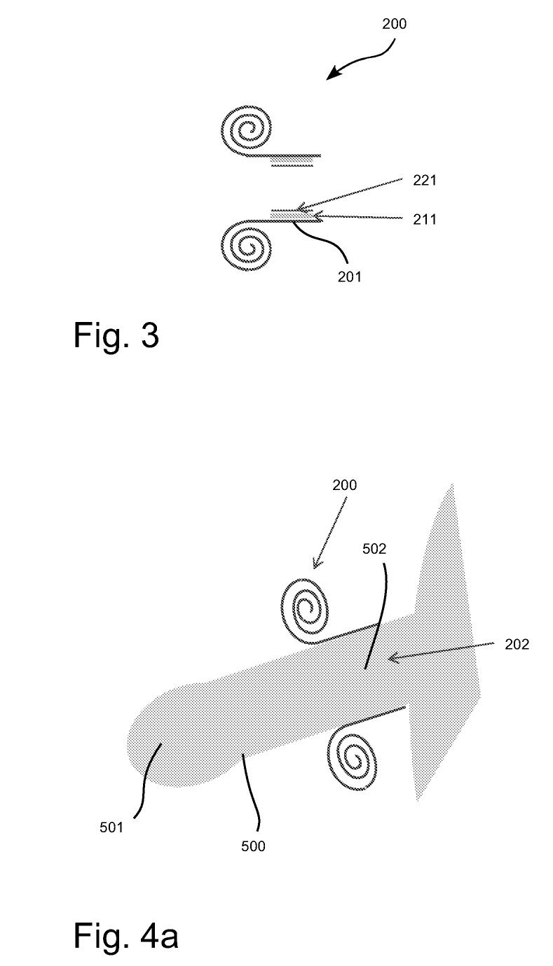

FIG. 3 discloses a cross sectional view of another example of an attachment member 200 with an adhesive 211 for adhesively attaching the attachment member to the surface of a penis. Furthermore, the attachment member comprises a protective film 221 for protecting the adhesive 211 prior to attachment of the attachment member to the surface of a penis.

FIG. 4a discloses a cross sectional view of an example of an attachment member 200 which has been applied on a penis 500. It is noted, that the attachment member is placed in an inwards position 502 on the penis and not on the glans penis 501. The attachment member has been applied to the penis by stretching the rolled up attachment member such that the inner diameter of the attachment member becomes greater than the outer diameter of the penis. The attachment member is then placed over the penis until it is located at an inwards position on the penis. It should be noted that since the attachment member has been stretched to a diameter larger than the diameter of the penis, the act of applying the attachment member is very easy and no force is applied to the penis itself.

Once the attachment member is in place at an inwards position on the penis, the attachment member is released and the attachment member contracts and establishes a firm connection at an inwards position on the penis. The attachment member is then ready to be unrolled in an outwards direction, i.e. in a direction away from the base of the penis. Furthermore it is to be noted that the second opening of the attachment member 202 has received an inwards position on the penis. It can also be noted that the first opening is rolled in the centre of the rolled up portion of the attachment member.

FIG. 4b discloses a schematic cross sectional view of the same example of an attachment member 200 on a penis 500 as in FIG. 4a. In FIG. 4b the attachment member has been unrolled allowing the first opening 203 to be in a position further outwards than the glans penis (501).

FIG. 4c discloses a schematic cross sectional view of the same example of an attachment member 200 on a penis 500 as in FIG. 4a and FIG. 4b. In FIG. 4c the outermost portion of the attachment member, in this specification called the connecting element, has been connected to a connecting member 231. The connecting element 241 of the attachment member 200 and the connecting member has, when applied, created a fluid tight seal. Furthermore it may be noted, that in the current example the connecting member comprises an adhesive 251 for supporting the engagement between the connecting element and the connecting member.

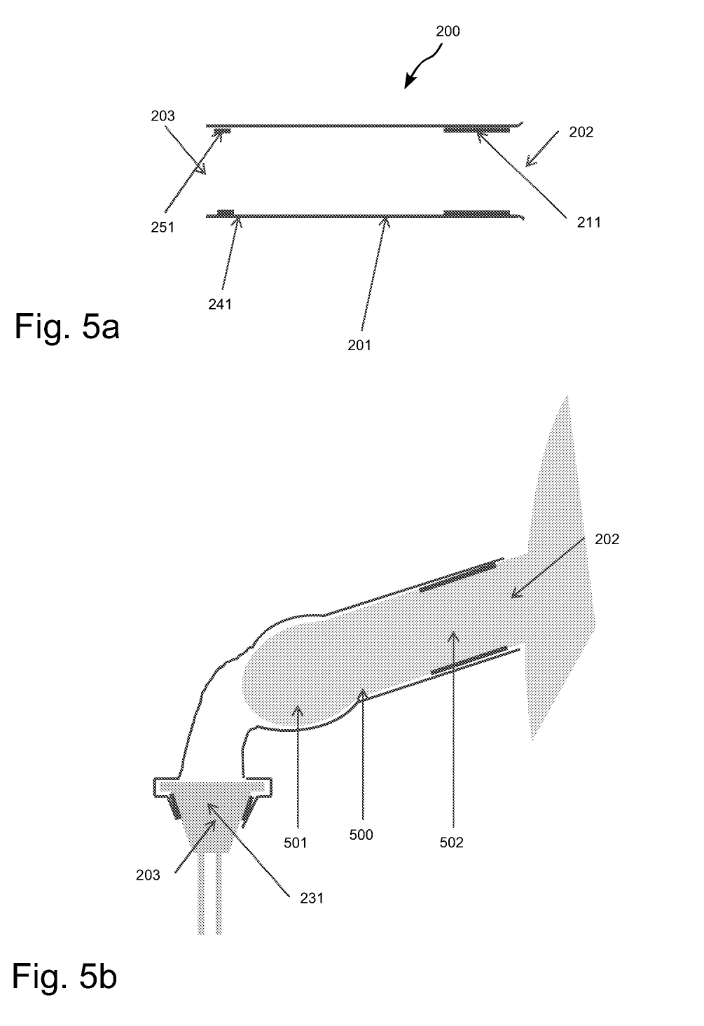

FIG. 5a discloses a cross sectional view of an example of an unrolled attachment member 200 according to the present invention. The unrolled attachment member has an elastic film material 201 and two openings, a second opening 202 for receiving a penis and a first opening 203 for receiving a connecting member from a urine container. This attachment member has a first adhesive 211 for supporting the connection between the attachment member and a penis. It also comprises a second adhesive 251 on the connecting element 241 for supporting the engagement between the attachment member and the urine container.

FIG. 5b discloses a cross sectional view of the same example of an attachment member 200 as in FIG. 5a, but now applied on a penis 500 and connected to a connecting member 231. It is noticed, that the elasticity of the elastic film material enables the elastic film material to conform around the glans penis 501 and supports the fastening of the attachment member on the penis.

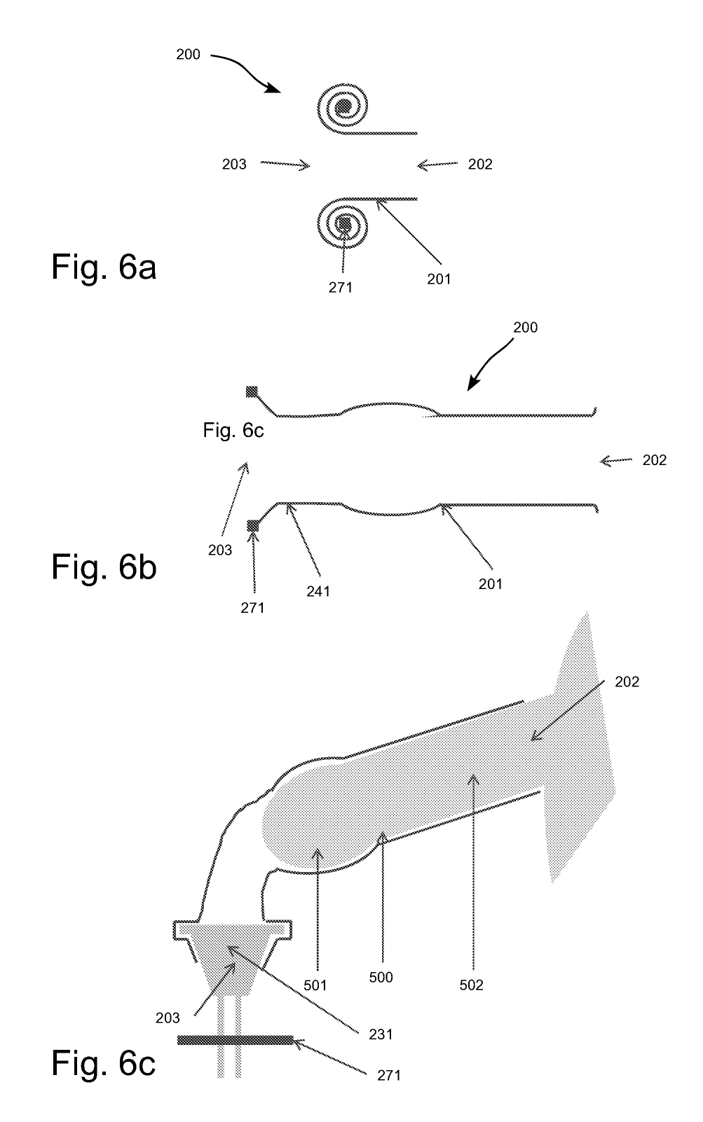

FIG. 6a discloses a schematic cross sectional view of an example of a rolled up attachment member 200 according to the present invention. The rolled attachment member comprises an elastic film material 201 and two openings, a second opening 202 for receiving a penis and a first opening 203 for receiving a urine container.

This attachment member comprises a ring shaped applicator 271 for supporting the positioning process of putting the member at an inwards position on a penis, the unrolling process in an outwards direction and the engagement between the attachment member and a urine container. The elastic film has been pre-rolled around the ring shaped application member. The ring shaped application member has an inner diameter larger than the diameter of the substantially cylindrical shaped elastic film and larger than the outer diameter of a penis. During the rolling process, the cylindrical diameter of the elastic film has been expanded from its original diameter to a diameter almost equal to the diameter of the ring shaped application member.

FIG. 6b discloses a schematic cross sectional view of the same example of an attachment member 200 as in FIG. 6a, but now in an unrolled state (for illustration purposes). It is noticed, that the elastic film material 201 is shaped like a penis shape with varying cylindrical diameter. Furthermore, it is noted that the elastic film, when unrolled from the application member, essentially contracts to its original size. It is also noticed, that the attachment member has a connecting element 241 for engagement with the urine collecting system.

FIG. 6c discloses a cross sectional view of the same example of an attachment member 200 as in FIGS. 6a and 6b, but now in an unrolled state on a penis 500 and connected to a connecting member 231. It is noticed, that the ring shaped application member 271, when the attachment member has been fully uncoiled, will separate from the attachment member and can be removed.

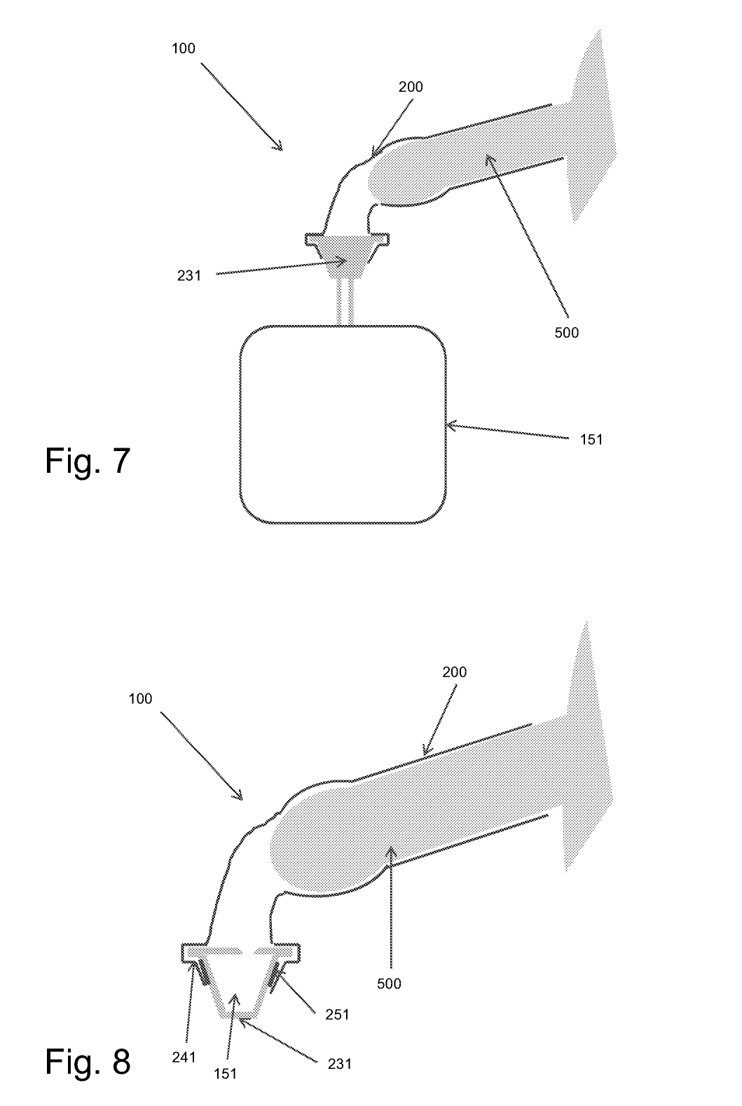

FIG. 7 discloses a cross sectional view of an example of a urine collecting system 100 according to the present invention, wherein the system is attached to a penis 500. The system comprises an attachment member 200, a connecting member 231 and a urine collector 151. The interior of the attachment member 200 is in fluid communication with the interior of the urine collector 151 via the connecting member 231.

FIG. 8 discloses a cross sectional view of an example of a urine collecting system 100 according to the present invention, wherein the system is attached to a penis 500. The system comprises an attachment member 200. It is noted that the connecting member 231 is integrally formed with the urine container 151. Furthermore, it is noted that the connecting member comprises an adhesive 251 for supporting the engagement of the connecting member 231 with the connecting element 241 on the attachment member. The urine container could comprise absorbent fibres for absorbing the urine within the container.

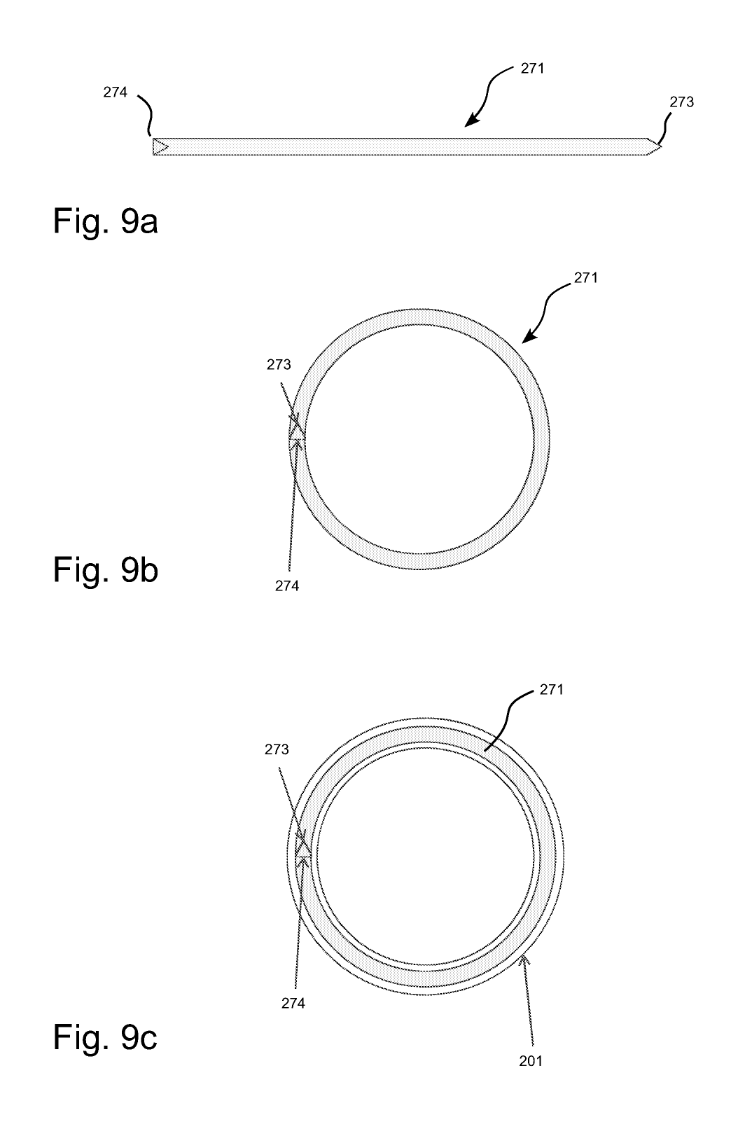

FIG. 9a discloses a cross sectional view of an example of an applicator 271 in the form of a shaped stick. The shaped stick has a receptor 273 and a connect form 274.

FIG. 9b discloses a top view of an example of an applicator 271 in the form of a shaped stick. The shaped stick has a receptor 273 and a connect form 274, wherein the receptor 273 has received the connect form 274 and formed a circular shape.

FIG. 9c discloses a top view of an example of an attachment member 200 comprising an applicator 271 in the form of a shaped stick. The shaped stick has a receptor 273 and a connect form 274, wherein the receptor 273 has received the connect form 274 and formed a circular shape. The elastic film material 201 of the attachment member is pre-rolled around the applicator 271. Such an applicator 271, as described in FIGS. 9a-9c will have a simple and even coil around its centre during uncoiling of the elastic film since the inner and outer lengths of the shaped stick are the same. This is in contrast to an injection moulded ring for example which will have a smaller inner length than outer length. When rolling a ring with different inner and outer lengths, the rolling process will be uneven.

FIG. 9 could also illustrate an applicator in the form of a spring. The spring should then be compressed during rolling of the substantially elastic film material. After rolling, when the spring is released, the spring will expand the diameter of the attachment member and thereby allow the member to initially be positioned on the penis rod.

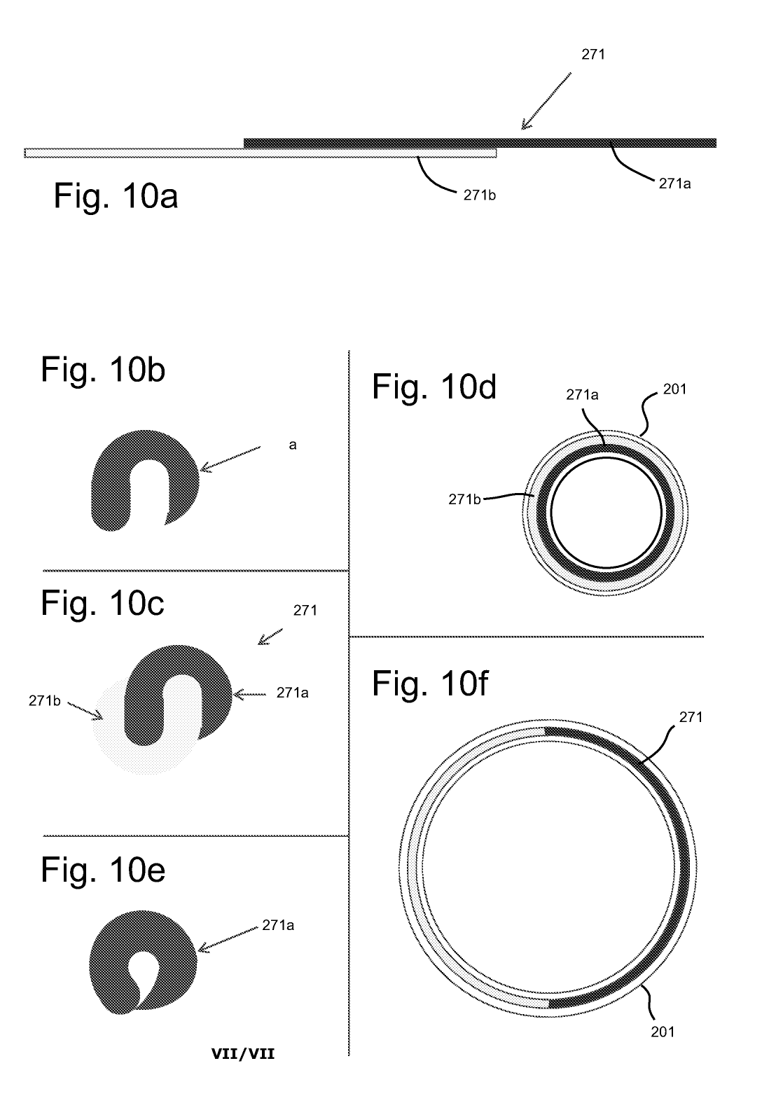

FIGS. 10a-f disclose different views of an applicator 271 comprising two shaped sticks 271a and 271b having approximate U-formed cross sections that can fit together and form a single shaped stick. The two shaped sticks are connected in a manner as shown in FIG. 10a, such that only the middle 50% of each shaped stick overlap. This shaped stick comprised of two separate shaped sticks is then bent to form a circular shape where the portions of the first and second shaped sticks which are not overlapping when straight (as shown in FIG. 10a) are then connected. The cross section of the connected shaped sticks is shown in FIG. 10c. When the applicator is pulled to expand the diameter, the two shaped sticks slide along their longitudinal axes with respect to each other and separate. As the U formed cross section is released from the other U-shaped cross section the U-form collapses due to the force from the pre-rolled attachment member as shown in FIG. 10e. Consequently, the expanded applicator cannot reversibly retract to its original size and it holds its new “stretched” configuration. The advantages of the applicator according to example 10 are that the attachment member can be rolled up on the applicator and stored in an approximately relaxed state and immediately prior to use be expanded. The problems with creep from soft TPEs are consequently solved.

FIG. 10a discloses a cross sectional view in the longitudinal direction of an example of an applicator 271 in the form of a shaped stick.

FIG. 10b discloses an enlarged circular cross sectional view of an example of U-formed shaped stick 271a of an applicator.

FIG. 10c discloses an enlarged circular cross sectional view of an example of two U-formed shaped sticks 271a and 271b fitted together forming an expandable applicator.

FIG. 10d discloses a top view of an attachment member 201 pre-rolled around an applicator 271 comprising two U-formed bended sticks 271a and 271b fitted together forming one expandable applicator.

FIG. 10e discloses an enlarged circular cross sectional view of an example of U-formed shaped stick 271a of an applicator, which has been expanded. Please note, that due to the retraction force of the elastic film material of the attachment member, the U-form of the stick 271a has collapsed so that the opposite U-formed stick 271b do not fit into 271a.

FIG. 10f discloses a top view of an attachment member 201 pre-rolled around an applicator 271 comprising two U-formed shaped sticks 271a and 271b which have been pulled to expand, why the U-formed sticks are collapsed. Consequently the applicator ring is expanded so it is possible to position the attachment member at an inwards position on a penis.

In the present example, the applicator is expandable to a diameter which is double that of its original diameter. If shapes and forms of shaped sticks comprising 3, 4 or 5 sticks were made, the applicator could similarly multiply its original diameter to 3, 4 or 5 fold its original diameter. Also smaller expansions are possible.

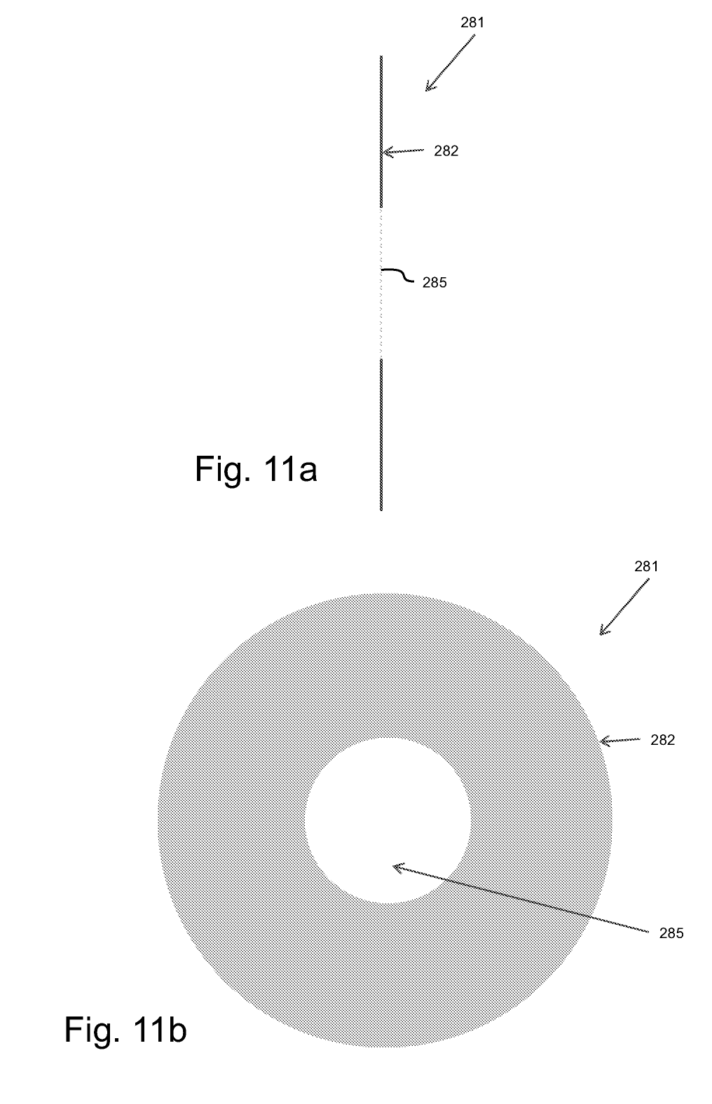

FIGS. 11a-b disclose different views of an example of a shield element 281 for shielding crotch hairs during the application of an attachment member. Such a shield member may comprise a hole or form 285 for receiving the penis.

FIG. 11a discloses a cross sectional view of an example of a shield element 281 with a flange portion 282 and a hole 285 for receiving a penis.

FIG. 11b discloses a front view of the example of the shield element 281 with a flange portion 282 and a hole 285 for receiving a penis. The shield element is put on the penis prior to application of the attachment member allowing the hole to receive the penis. The shield element will protect crotch hairs from being entangled with/in the application member during the un-rolling process of the attachment member. When the attachment member is applied, the shield element may be removed. The shield element could be made from different materials. One example is a thin sheet of fabric; another example is a thin sheet of paper or plastic.

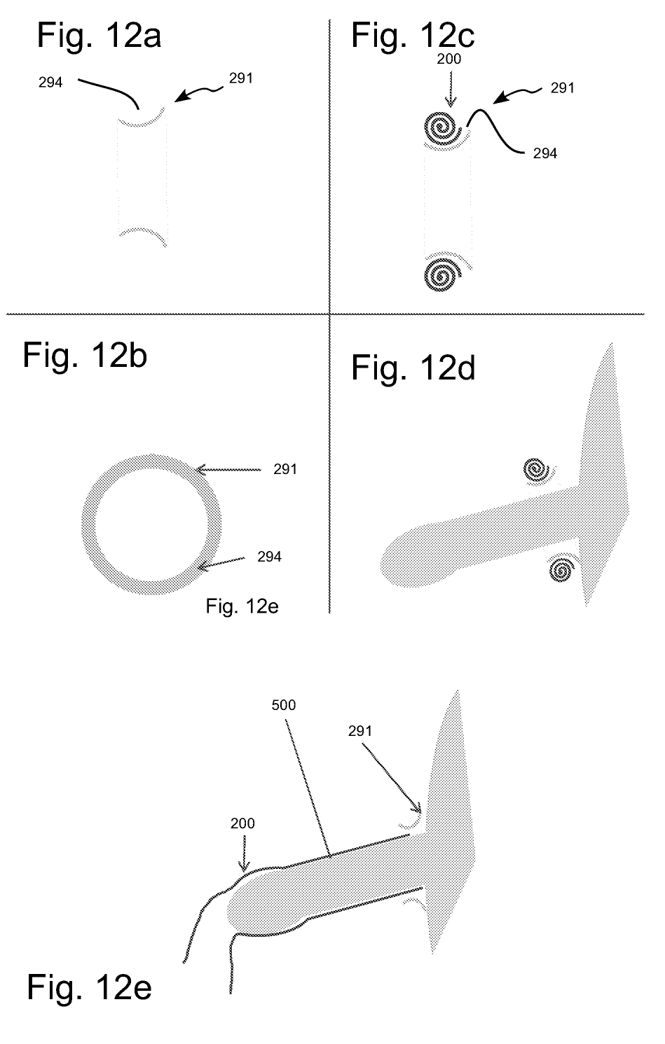

FIGS. 12a-e disclose different views of an example of an “external” applicator in the form of a separate member 291. In this example, the attachment member is not rolled around the applicator, but placed in a space 294 for receiving the attachment member 200. This type of applicator could be called an “external” applicator whereas the applicator of for example FIGS. 9 and 10 could be called an “internal” applicator.

FIG. 12a discloses a cross sectional view of an example of an applicator in the form of a separate member 291. The applicator 291 has a space 294 for receiving the attachment member.

FIG. 12b discloses a front view of the applicator of FIG. 12a. The space 294 for receiving the attachment member is shown on the applicator 291.

FIG. 12c discloses a cross sectional view of the applicator of FIGS. 12a-12b carrying an attachment member 200. The attachment member 200 is arranged in the space 294 of the applicator 291.

FIG. 12d discloses a cross sectional view of the applicator of FIG. 12a with an attachment member 200 positioned in an inwards position on a penis 500.

FIG. 12e discloses a cross sectional view of the applicator of FIG. 12a where the attachment member 200 has been rolled off the applicator and unto the penis.

When applying the attachment member on a penis using the applicator as shown in FIGS. 12a-12e, the pre-rolled attachment member 200 is placed in the space 294 of the applicator 291 by manually expanding the diameter of the attachment member and positioning it in the space. Then, the applicator is positioned at an inwards position on the penis, followed by rolling the attachment member in an outwards direction on the penis. When the attachment member is rolled on the penis, the applicator is removed by pulling the attachment member off the applicator and removing the applicator from the penis.

In the embodiment of FIGS. 12a-12e, the applicator has a fixed diameter which is larger than the outer diameter of the penis. However, one could also imagine an “external” applicator (not shown) where the attachment member is already mounted on the applicator during storage and then the applicator is expanded manually, thereby also causing the attachment member to expand, before placing the applicator in an inwards position on the penis. In one embodiment (not shown), the applicator comprises a ratchet mechanism which allows the applicator to expand in a first dimension, but not retract after it has been expanded.

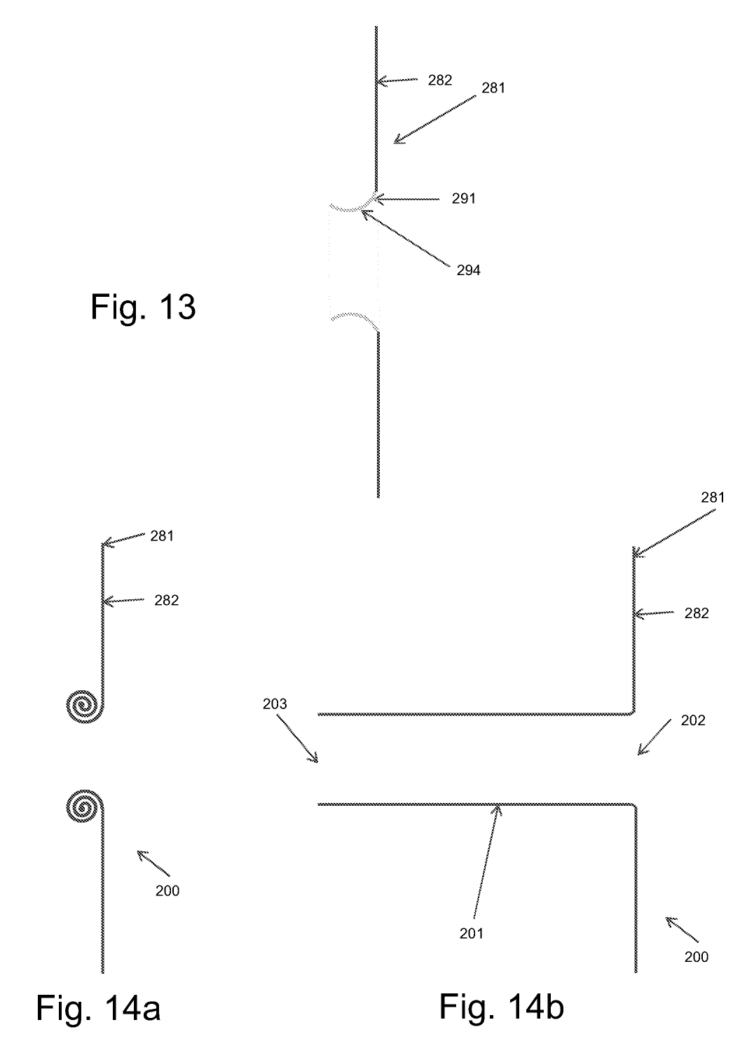

FIG. 13 discloses a cross sectional view of an “external” applicator in the form of a separate element 291, wherein the applicator comprise a shield element 281. The applicator 291 has a space 294 for receiving the attachment member. The shield element has a flange portion 282, for protecting crotch hair during the application of an attachment member. In this way, the person applying the attachment member can place the shield element against the body of the patient and then hold the applicator/shield element in position with the shield element while unrolling the attachment member.

FIGS. 14a-b disclose cross sectional views of an attachment member 200 comprising a shield element 281. The attachment member has a cylindrical portion 201 and openings 202 and 203.

FIG. 14a discloses a cross sectional view of an attachment member 200 comprising a shield element 281 with a flange portion 282. The attachment member is illustrated with a pre-rolled cylindrically shaped elastic film.

FIG. 14b discloses a cross sectional view of an attachment member 200 comprising a shield element 281 with a flange portion 282. The cylindrically shape elastic film portion 201 is illustrated un-rolled with the first and second opening 203 and 202.

The embodiment of FIGS. 14a and 14b could be manufactured such that the shield element and the attachment member are integrally formed such that the material of the shield element is the same as the material of the attachment member. However, one could also imagine an embodiment where the shield element was made from a first material and the attachment member was made from a second material and where the two materials are joined with an adhesive. One could also imagine an embodiment where the shield element could be separated from the attachment member after applying the attachment member on a penis. This could for example be provided by a line of perforations between the attachment member and the shield element.

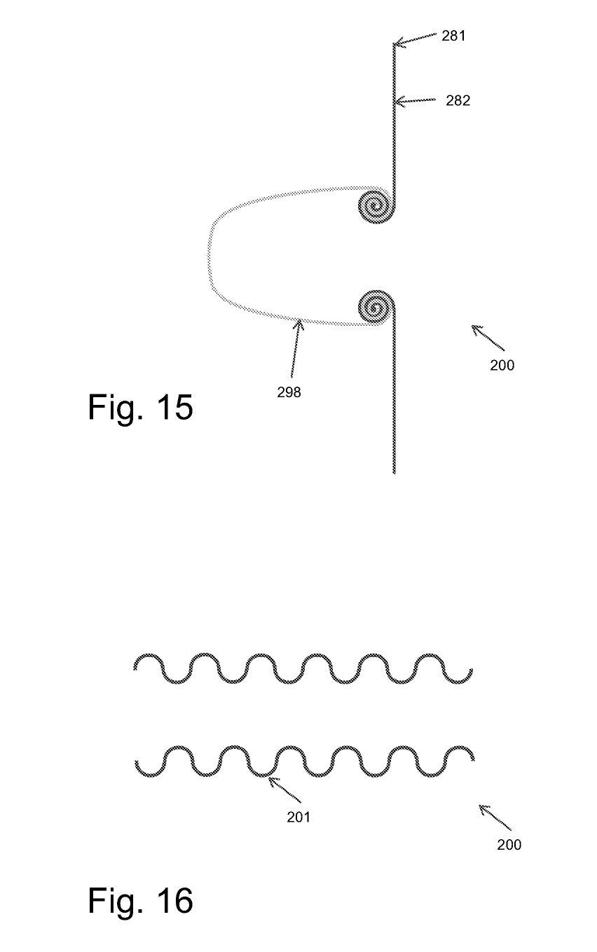

FIG. 15 discloses a cross sectional view of an attachment member 200 comprising a shield element 281 and an unrolling supportive element 298. The shield element comprises a flange portion 282. The unrolling supportive element 298 comprises two strips, which have been rolled into the attachment member and attached to each other. When unrolling the attachment member, the user simply pulls the unrolling supportive element 298 in an outwards direction on the penis while holding the base of the attachment member in place via the shield element 281.

FIG. 16 discloses a cross sectional view of an attachment member 200 wherein the essentially cylindrically shaped portion of elastic film 201 has a varying inner diameter. This will allow the attachment member to have areas with stronger attachment to the penis than others. It should be noted that when discussing the “inner diameter” of an attachment member such as the one disclosed in FIG. 1 where the inner diameter at different portions of the substantially cylindrical portion varies, the “inner diameter” as used in the claims should be understood as the average diameter of the portion(s) which engage(s) the penis when the attachment member is applied on a penis. For example, in the embodiment shown in FIG. 16, depending on the “amplitude” of the wave pattern, it could be that the inner portions are in contact with the penis while the outer portions of the wave are not in contact with the penis. Therefore, when defining the “inner diameter”, only those portions which are in contact with the penis should be considered.

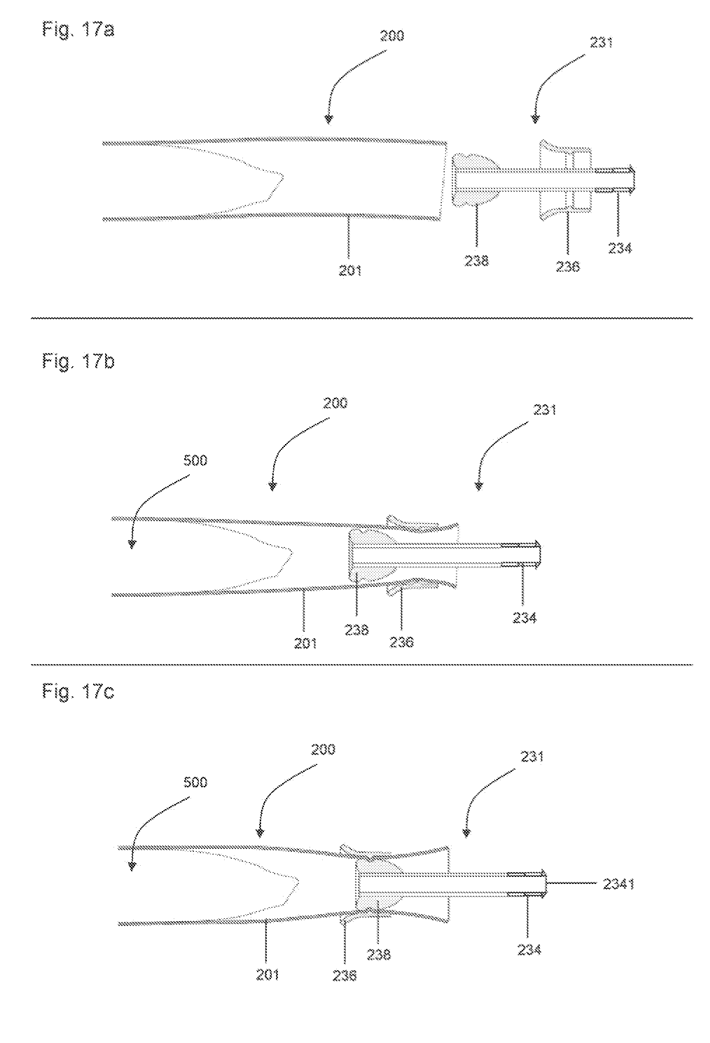

FIGS. 17a-c disclose cross sectional views of a kit of parts comprising an attachment member 200 with a cylindrically shaped elastic film 201 and a connecting member 231. The cylindrically shaped elastic film 201 is positioned on a penis 500. In this example, the connecting member 231 is in the form of a connector, more precisely in the form of a universal connector. At least a part of the universal connector 231 is soft and adapted to be connected to a urine bag 234. Urine bags are typically provided with a plastic connector at the end of a tube. Such connector has typical standard dimensions allowing a urine bag to fit any catheter. As the attachment member according to the present invention is not necessarily provided with a typical connector, the universal connector 231 will ensure that it is possible to fit the attachment member to a standard urine bag via the soft part 234.

FIG. 17a discloses a cross sectional view of a kit of parts comprising an attachment member 200 with a cylindrically shaped elastic film 201 and a universal connector 231, wherein the two parts are not connected. The universal connector comprises a hollow inserter 238 adapted to be inserted into the interior of the cylindrically shaped elastic film 201, a hollow sealing element 236 adapted to create a liquid tight seal between the inserter and the cylindrically shaped elastic film and a soft tubular part 234 adapted to be connected to any urine bag.

In FIG. 17b the connection between the cylindrically shaped elastic film 201 and the universal connector 231 is being established. The inserter 238 has been inserted into the cylindrically shaped elastic film 201, while the cylindrically shaped elastic film 201 has been drawn trough the sealing element 236.

In FIG. 17c the connection between the cylindrically shaped elastic film 201 and the universal connector 231 are complete. The sealing element 236 has been pushed onto the inserter 238 and thereby created a seal, bringing the interior of the cylindrically shaped elastic film 201 into fluid tight communication with the outlet 2341 of the soft tubular part 234.

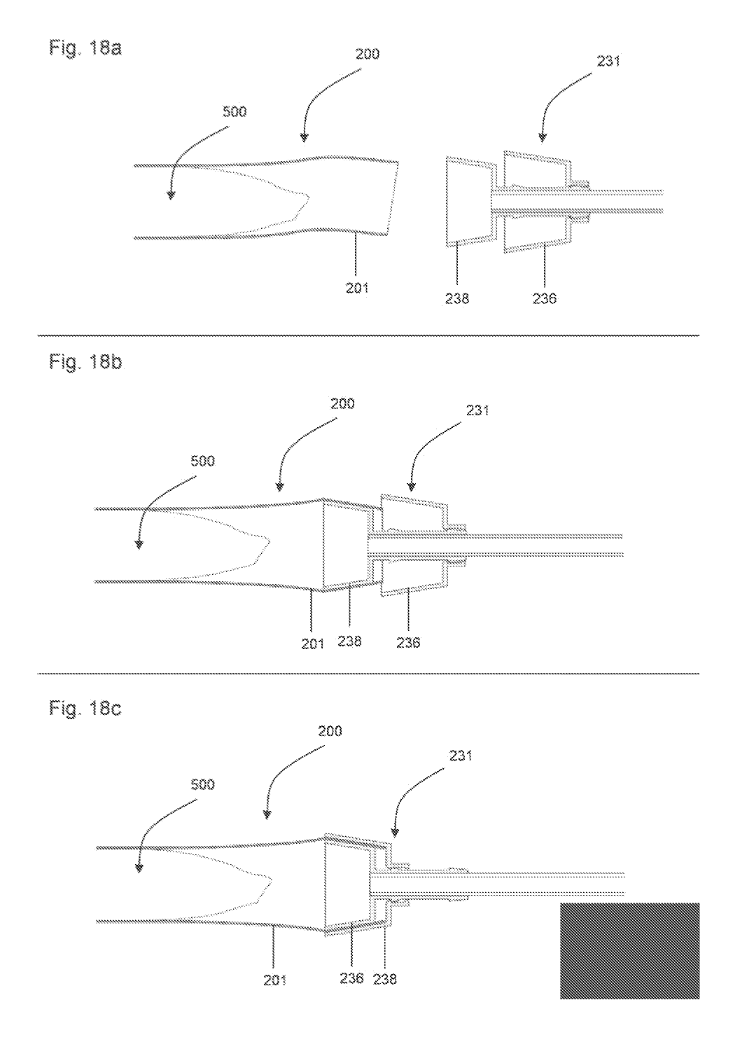

FIG. 18a-c disclose cross sectional views of a kit of parts comprising an attachment member 200 with a cylindrically shaped elastic film 201 and a connecting member 231. The cylindrically shaped elastic film 201 is positioned on a penis 500. The connecting member may be in fluid tight communication with the interior of a urine bag.

FIG. 18a discloses a cross sectional view of a kit of parts comprising an attachment member 200 with a cylindrically shaped elastic film 201 and a connector 231, wherein the two parts not are connected. The connector comprises a hollow inserter 238 adapted to be inserted into the interior of the cylindrically shaped elastic film 201, a hollow sealing element 236 adapted to create a liquid tight seal between the inserter and the cylindrically shaped elastic film.

In FIG. 18b the connection between the cylindrically shaped elastic film 201 and the connector 231 are being performed. The inserter 238 has been inserted into the cylindrically shaped elastic film 201.

In FIG. 18c the connection between the cylindrically shaped elastic film 201 and the connector 231 are complete. The sealing element 236 has been pushed onto the inserter 238 and thereby created a seal.

It should be noted that the current specification has disclosed a number of inventions which could form the basis for a number of divisional applications. For example, it can be noted that the claim scope of the current claim 1 has been limited to the embodiment of the attachment member having specific dimensions and being rolled up. However, a divisional application could be imagined which is directed to a more general attachment member, without any specific limitations as to its dimension of the attachment member, but instead combined with an applicator as discussed above into a kit of parts and further possibly comprising one or more of the other features disclosed in this application. Basis for an independent claim for this example divisional application can be found in current claim 18.

In another example, a divisional application could be considered which is limited to a more general attachment member, but where the limiting feature is the elasticity of the elastic film material of the attachment member limited to a specific flexibility/elasticity as mentioned in this specification. Basis for an independent claim for this divisional application can be found in current claim 17. Still a further possibility for a divisional application is an attachment member as disclosed herein, but without any limitations as to its size, in combination with a shield element. Basis for an independent claim for this divisional application can be found in current claim 19.

For these and other possible divisional applications, it should be clear to the person skilled in the art that all of the combinations as disclosed in the dependent claims and all of the features/elements of the embodiments disclosed in this specification related to the attachment member with a specific dimension could be combined with any of the above mentioned other independent claims.

1. An attachment member suitable for attaching a system to a male human being's penis, said system being suitable for collecting urine from a male human being, said attachment member comprising a substantially cylindrical portion which:
a. comprises an elastic film material,
b. has a first opening at a first end thereof and a second opening at the other end thereof,
c. is arranged such that at least the glans penis of a penis could be arranged inside the substantially cylindrical portion with the first opening being further from the base of the penis than the second opening, and
d. is at least partly rolled up to assume a ring like configuration in such a way that the first opening is located inside the rolled up portion and the second opening is located outside the rolled up portion characterized in that the inner diameter of the rolled up portion of the substantially cylindrical portion is less than 45 mm when the elastic film material of the attachment member is not stretched and not rolled up.

2. The attachment member according to claim 1, characterized in that the diameter of the second opening is greater than 20 mm when the second opening is not stretched.

3. The attachment member of claim 1, characterized in that the rolled up portion and the second opening are stretched such that the inner diameter of the rolled up portion and the diameter of the second opening are at least 10% greater than the inner diameter of the rolled up portion of the substantially cylindrical portion when the elastic film material of the substantially cylindrical portion is not stretched and not rolled up.

4. The attachment member of claim 1, characterized in that the elastic film material of the substantially cylindrical portion has a “modulus 100%” between 0.02 and 0.50.

5. A kit comprising the attachment member of claim 1 and an applicator which is arranged to stretch the rolled up portion of the substantially cylindrical portion such that the inner diameter of the rolled up portion is increased by at least 10% when compared to its non-stretched inner diameter.

6. The kit according to claim 5, characterized in that the applicator is arranged inside the rolled up portion of the substantially cylindrical portion.

7. The kit according to claim 5, characterized in that the applicator is arranged at least partly adjacent to the inner surface of the rolled up portion of the substantially cylindrical portion.

8. The kit of claim 5, characterized in that the applicator is arranged such that it can assume and hold at least two different configurations, a first configuration where the inner diameter of the rolled up portion is a first diameter and a second configuration where the rolled up portion has been stretched such that the inner diameter of the rolled up portion has been increased by at least 20% when compared to the first diameter.

9. The kit comprising the attachment member according to claim 5, characterized in that the kit further comprises a shield element arranged at the second opening of the substantially cylindrical portion of the attachment member, said shield element comprising a flange having an outer diameter which is at least 2 cm greater than the inner diameter of the rolled up portion of the substantially cylindrical portion when the rolled up portion is not rolled up and not stretched.

10. The kit according to claim 9, characterized in that the shield element is integrated with the attachment member or is integrated with the applicator.

11. A system for collecting urine from a male user, said system comprising
a. a urine container for collecting and/or storing urine during wear time, and
b. the attachment member according to claim 1.

12. A method of attaching an attachment member to a penis comprising the steps of:
a. providing an attachment member comprising a substantially cylindrical portion which:
i. comprises an elastic film material,
ii. has a first opening at a first end thereof and a second opening at the other end thereof,
iii. is arranged such that at least the glans penis of a penis could be arranged inside the substantially cylindrical portion with the first opening being further from the base of the penis than the second opening, and
iv. is at least partly rolled up to assume a ring like configuration in such a way that the first opening is located inside the rolled up portion and the second opening is located outside the rolled up portion,
b. stretching the rolled up portion and the second opening such that the inner diameter of the rolled up portion and the diameter of the second opening are at least 1% greater than the inner diameter of the rolled up portion of the substantially cylindrical portion when the elastic film material of the substantially cylindrical portion is not stretched and not rolled up,
c. positioning the attachment member on the outer surface of the penis rod at a position between the glans penis and the base of the penis; and
d. unrolling at least a part of the attachment member in a direction along the penis and away from the base of the penis.

13. A method according to claim 12, further comprising the step of releasing the stretching force applied to the attachment member and allowing it to retract, after positioning the attachment member on the outer surface of the penis.

14. A method of claim 12, further comprising the steps of determining the approximate outer diameter of the penis and providing an attachment member which is sized appropriately for the approximated outer diameter of the penis.

15. The attachment member according to claim 1.

16. The method of an elastic film material in the manufacture of the attachment member according to claim 1.

17. The attachment member suitable for attaching a system for collecting urine from a male human being to a male human being's penis, the attachment member comprising a substantially cylindrical portion comprising an elastic film material, said substantially cylindrical portion having a first opening at a first end thereof and a second opening at the other end thereof and where the attachment member is arranged such that at least the glans penis of a penis could be arranged inside the substantially cylindrical portion with the first opening being further from the base of the penis than the second opening, characterized in that the elastic film material of the substantially cylindrical portion has a “modulus 100%” between 0.02 and 0.50.

18. The kit comprising
a. the attachment member suitable for attaching a system for collecting urine from a male human being to a male human being's penis, the attachment member comprising a substantially cylindrical portion:
i. which comprises an elastic film material,
ii. which has a first opening at a first end thereof and a second opening at the other end thereof,
iii. which is arranged such that at least the glans penis of a penis could be arranged inside the substantially cylindrical portion with the first opening being further from the base of the penis than the second opening, and
iv. which is at least partly rolled up to assume a ring like configuration in such a way that the first opening is located inside the rolled up portion and the second opening is located outside the rolled up portion, and
b. an applicator which is arranged to stretch the rolled up portion such that the inner diameter of the rolled up portion is increased by at least 10% over its non-stretched inner diameter.

19. The attachment member suitable for attaching a system for collecting urine from a male human being to a male human being's penis, the attachment member comprising a substantially cylindrical portion comprising an elastic film material, said substantially cylindrical portion having a first opening at a first end thereof and a second opening at the other end thereof and where the attachment member is arranged such that at least the glans penis of a penis could be arranged inside the substantially cylindrical portion with the first opening being further from the base of the penis than the second opening, characterized in that the attachment member further comprises a shield element arranged at the second opening of the substantially cylindrical portion.
